(19) United States
(12) Patent Application Publication
(10) Pub. No.: US 2014/0277169 A1
(43) Pub. Date: Sep. 18, 2014

(54) Vertebral Implant

(71) Applicants: Nadi Salah Hibri, San Antonio, TX (US); James Douglas Lutz, San Antonio, TX (US)

(72) Inventors: Nadi Salah Hibri, San Antonio, TX (US); James Douglas Lutz, San Antonio, TX (US)

(21) Appl. No.: 13/831,261

(22) Filed: Mar. 14, 2013

(51) Int. Cl. A61B 17/70; A61B 17/88

(52) U.S. Cl. CPC A61B 17/7098; A61B 17/7094; A61B 17/8855
USPC 606/279; 606/93

(57) ABSTRACT

An exemplary implantable device may be used to strengthen and stabilize a human vertebra. The device may include a cannula and support casing. In general, the device may include various interchangeable structures within cannula and support casing. After implantation, the device remains in the vertebral body of the vertebra in order to provide support and stability. Portions of the device remains firmly anchored in the pedicle, preventing the device from shifting. Portions of the device may be additionally secured external to the vertebra to provide additional stability.

### TECHNICAL FIELD

This invention relates to medical devices, and more particularly to implants for strengthening and stabilizing a spine.

### BACKGROUND

The human vertebral column is a vital part of the human physiology that houses and protects the spinal cord, and provides structural support for the body. In a typical human, the vertebral column is made up of twenty-four articulating vertebrae and nine fused vertebrae. While variations exist between each vertebra depending on its location and region, vertebrae generally consist of a body, pedicles, a lamina, a spinous process, transverse processes, facet joints, and a spinal canal, each of which play a pivotal role in providing the overall supportive and protective functionality of the vertebral column. Of these features, the vertebral body is of particular importance in providing support. The vertebral body is the largest portion of the vertebra, provides an attachment point of intervertebral discs, protects the spinal cord, and bears the majority of the load of the vertebra.

Due to trauma or disease, such as osteoporosis, vertebra may develop structural weaknesses, particularly in the vertebral bodies. These weaknesses may leave the spinal column vulnerable to compression fractures and susceptible to uneven force transference between vertebrae, resulting in acute or chronic pain, a loss of body height, as well as a reduction in mobility.

Kyphoplasty is often used to treat this condition. In this procedure, a balloon is inflated into cancellous bone to form a cavity. The balloon is deflated and removed. Flowable cement (such as methyl methacrylate) is then injected into the cavity, restoring the original height of the vertebral body. However, several difficulties are inherent this process.

First, stabilizing the bone by injecting cement into a cavity created in the bone is difficult because of persistent motion and poor anchoring of the cement to the fragile trabecular bone. Pseudoarthrosis is a common occurrence and this is often associated with persistent pain. Vertebral instability, further loss of vertebral height and increase in kyphosis or lateral angulation, retropulsion of a posterior vertebral fragment into the spinal canal, insufficiency fractures of the pedicles, and other forms of instability are often demonstrated on imaging.

Second, there is often an unacceptable high rate of leakage of cement into the paraspinal tissues, including leakage into the spinal canal and neural foramina, adjacent disc, or into the veins. This is due to the presence of subtle fractures involving the cortical bone of the vertebral body and end plates.

Third, because of fear of cement leakage, adequate intravertebral pressure cannot be achieved, resulting in failure to restore vertebral height and angulation.

Ideally, an alternative procedure should be developed to address these concerns. An alternative vertebral stabilization procedure should address the issues of poor implant anchoring, cement leakage, and inadequate restoration of height and angulation. Implant devices used in this procedure should be compatible with existing percutaneous surgical procedures, such that the implant is minimally invasive during insertion and use. The device should also be controllably expandable in one or more specific dimensions, such that the device can be deployed to regions with specific dimensional restrictions. The device should also remain stable within the implanted region, such that it does not move or deform undesirably after implantation. The device should also be implantable without requiring numerous additional tools, such that the number of tools that must be simultaneously inserted is reduced.

### SUMMARY

This specification describes technologies relating to the strengthening and stabilization of the spine. Implementations of the technology described herein comprise a surgical device that is implanted through a small surgical incision into a portion of a human vertebra, and a method by which the device is used to strengthen and stabilize a vertebra.

Various implementations of the present invention provide benefits that are desirable for surgical applications. The device is compatible with existing percutaneous surgical procedures, as it can be inserted and fixably implanted into the body through a single small incision with minimal damage to healthy surrounding tissue. The device is also controllably expandable in one or more specific dimensions. As such, the device can be deployed to regions with specific dimensions restrictions, and without disturbing adjacent regions of healthy tissue. The device can also be securely attached to the vertebra, such that it will not shift after implantation. The device is also implantable without requiring numerous additional tools, such that the number of tools that must be simultaneously inserted is reduced and damage to healthy tissue is minimized.

In an example implementation of the present invention an apparatus comprises: an axially extending cannula, which further comprises a first end, forming a first access orifice along the axial extension of the cannula, a second end having an internally threaded portion, forming a second access orifice along the axial extension of the cannula, and a tubular channel extending from the first access orifice to the second access orifice; an axially extending support device further comprising a first end having an externally threaded portion, forming a first access orifice along the axial extension of the support device; and a second end, forming a second access orifice along the axial extension of the support device; a tubular channel extending from the first access orifice to the second access orifice; wherein the threaded portion of the first end of the support device is adapted to cooperatively fit into the threaded portion of the second end of the cannula; and wherein an internal diameter of the support device is smaller than an internal diameter of the cannula; and an implant cartridge further comprising: a tubular liner element having a first portion, forming a first access orifice, and a second portion, forming a second access orifice, where an external diameter of the first portion corresponds to the internal diameter of the cannula, and an external diameter of the second portion corresponds to the internal diameter of the support device; and an inflatable structure within the liner element, having a filling aperture; wherein the implant cartridge is adapted to fit within the cannula and the support device when the cannula and the support device are assembled; wherein when the cannula, support device, and implant cartridge are assembled, filling the inflatable structure with a material expands the balloon out of the second access hole of the support device.

In another example embodiment of the present invention, an apparatus comprises: an axially extending tubular cannula having an internally threaded end portion; an axially extending tubular support device having an externally threaded end portion; an inflatable structure adapted to fit within the cannula and the support device when the apparatus is assembled; wherein when the apparatus is assembled, the threaded end portion of the support device couples into the threaded end portion of the cannula; and wherein filling the inflatable structure with a material expands the inflatable structure out of the support device.

In yet another example embodiment of the present invention, implementations may include one or more of the following features. An internal diameter of the support element is smaller than an internal diameter of the cannula. A delivery device adapted to transfer a material from the delivery device to the inflatable structure. A guide device having a pointed end, adapted to fit through the cannula and through the support device when cannula and support device are assembled. The apparatus comprises a surgically compatible material. Portions of the apparatus comprise a biocompatible material. Portions of the apparatus comprise a radiopaque material. The apparatus is adapted to insert into a vertebra. The apparatus is adapted to implant the inflatable structure within a vertebra. The inflatable structure strengthens the vertebra when inflated. The cannula may be released from the access device after the apparatus is inserted into a vertebra. The material may be poly methyl mecratylate, silicone, bone cement, epoxy, acrylic, or a combination thereof. The material cures or reacts within the inflatable structure such that the substance changes phase, tensile strength, density, size or other physical properties upon curing or reacting.

In yet another example embodiment of the present invention, a method comprises: attaching a cannula to a support device by screwing a threaded portion of the cannula to a threaded portion of the support device; inserting the support device into the body inserting an implant cartridge into the cannula and support device, the implant cartridge comprising an inflatable structure; inflating the inflatable structure such that a portion of the inflatable structure exits the support device and into the body; removing the cannula from the support device by unscrewing the threaded portion of the cannula from the threaded portion of the support device; wherein after removing the cannula, the support device and the inflatable structure remain in the body. The method may further comprise inserting a guide device into the cannula and support device prior to inserting the support device into the body, and removing the guide device after inserting the support device into the body.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a portion of a human vertebral column.

FIG. 2 is a cross-sectional view of a human vertebra.

FIGS. 3A-C are perspective views of embodiments of a surgical implant.

FIGS. 4A-F illustrate an example use of an exemplary surgical implant.

FIGS. 5A-D are cross sectional views of embodiments of a surgical implant.

FIGS. 6A-B illustrate example embodiments of the present invention.

FIG. 7 illustrates an example embodiment of the present invention.

FIGS. 8A-B illustrate example embodiments of the present invention.

FIG. 9 illustrates an example embodiment of the present invention.

FIGS. 10A-B illustrate example embodiments of the present invention.

FIGS. 11A-H illustrate an example embodiment of the present invention.

Like reference symbols in the various drawings indicate like elements.

### DETAILED DESCRIPTION

The following description is of one exemplary embodiment of the invention. The description is not to be taken in a limiting sense, but is made for the purpose of illustrating the general principles of the invention. Various inventive features are described below that can each be used independently of one another or in combination with other features.

Broadly, an embodiment of the invention provides a surgical implant device for strengthening and stabilizing a human vertebra, and a system for implanting the device in a human vertebra.

FIGS. 1-2 illustrate a portion of a typical human vertebral column. A vertebral column 100 is made up of several vertebrae 102, 104, and 106 separated by intervertebral discs 108 and 110. Vertebra 102 includes vertebral body 202, pedicles 204 and 206, spinous process 208, and spinal canal 210.

FIG. 3 illustrates an exemplary implantable device 300 used to strengthen and stabilize human vertebra 102. Device 300 includes cannula 302 and support casing 304. In general, device 300 may include various interchangeable structures within cannula 302 and support casing 304. In some embodiments, device 300 includes guide pin 306 within cannula 302 and support casing 304, for example, as illustrated in FIG. 3A. In some embodiments, guide pin 306 is slideably interchangeable with inflatable cartridge 308, as illustrated in FIGS. 3B and 3C. In some embodiments, inflatable cartridge 308 includes an inflatable structure 310. Inflatable structure 310 may have a deflated form (FIG. 3B) and an inflated form (FIG. 3C).

FIG. 4 depicts an exemplary usage of device 300. Referring to FIG. 4A, device 300, including guide pin 306, is inserted through the skin and positioned such that guide pin 306 is laterally positioned against pedicle 204. Referring to FIG. 4B, force is applied to device 300, piercing device 300 through the pedicle and into vertebral body 202. In this position, a portion 404 of support casing 304 remains outside of vertebra 102. Guide pin 306 is removed, while cannula 302 and support casing 304 remain in an inserted position (FIG. 4C), and inflatable cartridge 308 is inserted into cannula 302 and support casing 304 (FIG. 4D). Referring to FIG. 4E, inflatable cartridge 308 is filled with material 402, expanding inflatable structure 310 out of support casing 304 and into vertebral body 202. Referring to FIG. 4F, cannula 302 is separated from support casing 304 and removed from the body. Support casing 304 and inflatable cartridge 308 remain within the vertebra 102. Before, during and after insertion of device 300, spinal canal 210 remains undisturbed.

After implantation, inflatable structure 310 remains in vertebral body 202 in order to provide support and stability. Support casing 304 remains firmly anchored in the pedicle, preventing inflatable structure 310 from shifting. Portion 404 of support casing 304 may be additionally secured external to vertebra 102 to provide additional stability to support casing 304, inflatable cartridge 308, and inflatable structure 310.

Additional devices 300 may also be implanted into the body simultaneously or in succession. For example, a second device 300 may be implanted through pedicle 206 such that two inflatable structures 310 are deployed within the vertebral body 202.

FIG. 5 illustrates cross-sections of exemplary embodiments of device 300. Referring to FIG. 5A, cannula 302 is depicted as an axially extending tube having an internally threaded portion 502. Support casing 304 is illustrated as an axially extending tube having an externally threaded portion 504. Threaded portions 502 and 504 may be coupled, connecting cannula 302 and support casing 304, and defining channel 506 between access holes 508 and 510. The inner diameter of supporting casing 304 is smaller than the inner diameter of cannula 302, such that stop 508 is defined within channel 506. Edge 516 is defined at the end of support casing 304, and may be sharpened or beveled so that device 300 may be more easily inserted into tissue.

Cannula 302 may be separated from support casing 304 by decoupling the threaded portions, such as by rotating the cannula radially relative to the support casing 304. In this manner, cannula 302 may be separated from support casing 304 after implantation, such that support casing 304 remains within vertebra 102 while cannula 302 is removed from the body.

In some embodiments, cannula 302 and support casing 304 are attached by mechanisms other than corresponding threaded portions. In some embodiments, cannula 302 and support casing 304 are joined by corresponding clasps, tabs, or latches. In some embodiments, cannula 302 and support casing 304 are joined by adhesive. In some embodiments, cannula 302 and support casing 304 are permanently connected, such as through a weld or an adhesive, or manufactured as a single piece.

In some embodiments, guide pin 306 may be placed within cannula 302 and support casing 304. Guide pin 306 is illustrated as generally cylindrical with pointed end 514. Guide pin 306 includes a portion having an outer diameter corresponding to the inner diameter of support casing 304, and a portion having an outer diameter corresponding to the inner diameter of cannula 302, such that guide pin 306 may be slideably inserted into channel 506 from access hole 510. Guide pin 306 is set by stop 512, such that end 514 protrudes from access hole 508 when at the set position.

End 514 of guide pin 306 is illustrated as a conical shape, but may be of other shapes. In some embodiments, end 514 is of a rounded shape, a flat shape, or a beveled shape. End 514 may form a point along the central axis of guide pin 306, or may be formed at a different point. End 514 may also incorporate more complex structures, such as screws, protrusions, or grooves. Guide pin 306 may be releasably fixed at the set position, such that it will not move until it is released. In some embodiments, guide pin 306 is fixed to cannula 302 or support casing 304 using a pin, a tab, a friction cuff, or other such fastening mechanism.

Referring to FIG. 5B, guide pin 306 may be slideably removed and replaced with inflatable cartridge 308. Inflatable cartridge 308 includes liner 518 and inflatable structure 310. Liner 518 is illustrated as generally tubular, with a portion having an outer diameter corresponding to the inner diameter of support casing 304, and a portion having an outer diameter corresponding to the inner diameter of cannula 302. Cartridge 308 may be slideably inserted within cannula 302 and support casing 304 from access hole 510, and set by stop 512. Inflatable structure 310 is positioned within liner 518 and secured at affixing point 522. Inflatable structure 310 defines a filling aperture 520, through which material is passed into inflatable structure 310.

In FIG. 5B, inflatable structure 310 is illustrated in a deflated form. Referring to FIG. 5C, material 402 may be placed into inflatable structure 310 through filling aperture 520, resulting in expansion of inflatable structure 310 out access hole 508. Inflatable structure 310 may dimensionally expand based upon the volume of material 402 placed into it, illustrated for example as regions a, b, and c. Inflatable structure 310 may be made of an elastomeric material, such that it may stretch when filled. Material 402 may be retained in inflatable structure 310 through the use of a self-sealing valve or plug.

In some embodiments, inflatable structure 310 may be adapted such that filling it with material 402 will cause it to expand substantially in one or more pre-determined dimensions, but not in one or more other pre-determined dimensions. Thus, inflatable structure 310 can expand to fit a particular region as desired. Inflatable structure 310 may contain particular features, such as a folds or differentially elastomeric regions, such that expansion may occur in pre-determined directions.

Material 402 may be placed into inflatable structure 310 using a filling tool 526. Referring to FIGS. 5C and 5D, filling tool 526 is depicted as generally tubular and is adapted to slideably insert into access hole 510 into cannula 302. A portion 528 of filling tool 526 is shaped to allow filling aperture 530 to abut filling aperture 520. Filling tool 526 contains material 402, which may be transferred from filling tool 526 to inflatable structure 310 when filling apertures 530 and 520 are abutted. The transfer of material 402 may be caused by pressure, such as by squeezing filling tool 526, by using a pumping mechanism, or by using of a syringe-type mechanism.

Material 402 may be a liquid, gel, or quasi-solid material. Material 402 may cure or react within the inflatable structure 310, such that the substance changes phase, tensile strength, density, size, or other physical properties upon curing or reacting. Material 402 may be poly methyl methacrylate, silicone, bone cement, epoxy, acrylic or other such material. Material 402 may also contain imaging contrast agents, such as radiopaque materials or paramagnetic materials, such that imaging contrast is enhanced during commonly used medical imaging techniques.

Portions of device 300, such as cannula 302, supporting casing 304, guide pin 306, and inflatable cartridge 308 are illustrated with a circular cross-section. In other embodiments, one or more of these portions may alternatively have a non-circular cross-section, for example a square, oval, polygon, or irregular shape.

Portions of device 300 may be made of various materials, such as metal, plastic, acrylic, or glass. Device 300 may be made of surgically compatible materials, such that they can be safely used in a sterile environment. Portions of device 300 may be made of biocompatible materials, such that they may be safely implanted into the body without risk of immune response. Some portions of device 300 may be made of a radiopaque material, such that they provide imaging contrast during x-ray or fluoroscopic procedures. Device 300 may be made of non-ferrous materials, such that they are usable in conjunction with magnetic resonance imaging. Portions of device 300 may be made of paramagnetic or super paramagnetic materials, such that they provide imaging contrast during MRI.

In some embodiments, components of device 300 are detachably connected, such that each of the components may be independently removed, cleaned, and replaced. In some embodiments, portions of device 300 are designed to be disposable, while other portions are designed to be repeatedly reused.

The device may also be implanted into other portions of the body, and is not limited only to the vertebral body of a vertebra. Other locations include bones such as the femur, humerus, pelvis, or any other bone within the body.

In some embodiments, support casing 304 may include one or more apertures 602 to further increase the stability of device 300 after it has been implanted. An example embodiment is illustrated in FIG. 6A, where a device 300 includes several apertures 602 running the length of support casing 304. Apertures 602 may be of various forms, such as circular, ovular, or nearly-annular. Apertures 602 may be arranged in various ways on supporting casing 304, for example evenly spaced around the periphery of support casing 304 or in a particular defined pattern. As illustrated in FIG. 6B, tool 300, including support casing 304, may be inserted into a sharpened cannula 606 for surgical insertion into the body. When expanded, inflatable structure 310 pushes out of apertures 602 and out of supporting casing 304, forming multiple protuberances 604 that may contact external surfaces to anchor deployed device 300 to the surrounding material. An example implementation is illustrated in FIG. 7. Device 300 is first inserted into vertebra 102 through pedicle 204. If a cannula 606 was used to insert device 300, it is removed while device 300 remains inserted in vertebra 102. Device 300 is then inflated, pushing inflatable structure 310 out of apertures 602, forming protuberances 604 that come into contact with pedicle 204. After inflation, device 300 remains firmly anchored to vertebra 102 through frictional contact from protuberances 604.

In some embodiments, filing tool 526 may be releasably attached to inflation cartridge 308. This may be implemented, for example, through a threaded portion 802 on filing tool 526, and a corresponding threaded portion 804 on inflation cartridge 308, as illustrated in FIG. 8. Filing tool 526 may be connected to inflation cartridge 308 by turning either element axially to engage the threaded portions, or may be released by turning in the opposite direction to disengage the threaded portions. In some embodiments, threaded portions 802 and 804 may be defined on corresponding oblique angled edges to ensure that the tools can only be connected in the correct orientation. Filing tool 526 and inflation cartridge 308 are illustrated in a connected configuration in FIG. 8A, and in a disconnected configuration in FIG. 8B.

In some embodiments, an obturator 806 may be used to drive the contents of filling tool 526 out of filling tool 526. An example embodiment of obturator 806 is illustrated in FIG. 8C. Obturator 806 is generally cylindrical in shape with a pointed end 808 shape to correspond with portion 802 of filling tool 526. A user may insert obturator 806 into filling tool 526 and apply pressure to drive the contents of filling tool 526 completely into inflatable cartridge 308. This also ensures that filling tool 526 may be cleanly separated from inflatable cartridge 308 after filling.

In some embodiments, support casing 304 may also include several anchors 902, as illustrated in FIG. 9. Anchors 902 are pointed protrusions that extend radially outward from support casing 304, and may be of various shapes, such as a conical or a rounded shape, as illustrated in FIG. 9B. Anchors 902 may be distributed evenly along support casing 304, or may be distributed in a particular pattern.

In some embodiments, support casing 304 is of an expandable design, such that it may be partially collapsible in the radial direction, but may expand radially when outward force is applied to the interior surface 904 of support casing 304. In this manner, support casing 304 may be radially collapsed to a width of W to slideably fit into a delivery cannula 1002, as illustrated in FIG. 10A, then expanded to a width of W+W₁ by a expansion balloon 1004 when no longer contained in cannula 1002, as illustrated in FIG. 10B. In these embodiments, support casing 304 may be made of memory materials, such as nitinol, that expand to a pre-defined shape when no external force is applied to it, but may be compressed, such as to fit into delivery cannula 1002. Support casing 304 may alternatively be made of fabric, a polymer, a composite material, memory metal, or a combination of two or more of these materials. For example, support casing 304 may be formed by a fabric with memory metal supports woven into the fabric.

Supporting casing 304 and delivery cannula 1002 are depicted as tubular with a generally circular cross section, but may instead have an ovular, elliptical, polygonal, or irregular cross section. Expansion balloon 1004 is shaped to slideably insert into support casing 304, and is depicted as a generally cylindrical balloon that expands radially when inflated. Expansion balloon 1004 includes an input valve 1006 through which gas may be inserted or removed from expansion balloon 1004.

Anchors 902 may be used to securely attach support casing 304 to surrounding structures after implantation into vertebra 102. An example implementation is illustrated in FIG. 11. Referring to FIG. 11A, a guide pin 1102 is slideably inserted into delivery cannula 1002, and guide pin 1102 and delivery cannula 1002 are inserted through the skin and positioned that such that guide pin 1102 is laterally positioned against pedicle 1104 of vertebra 102. Guide pin 1102 is adapted to slideably insert into delivery cannula 1002, but is otherwise generally similar to other guide pins, as described above.

Referring to FIG. 11B, force is applied to device 300, piercing guide pin 1102 and delivery cannula 1002 through the pedicle and into vertebral body 1106.

Referring to FIG. 11C, guide pin 1102 is slideably removed from delivery cannula 1002. Expansion balloon 1004 is slideably inserted into support casing 304, and both expansion balloon 1004 and support casing 304 are slideably inserted into delivery cannula 1002.

Referring to FIG. 11D, delivery cannula 1002 is slideably removed from vertebra 102, leaving a channel 1108. Support casing 304 and balloon 1004 remain in channel 1108.

Referring to FIG. 11E, expansion balloon 1004 is inflated, expanding support casing 304 until anchors 902 abut, then pierce, pedicle 1104. This firmly anchors support casing 304 into pedicle 1104 and vertebra 102 and prevents further movement of support casing 304.

Referring to FIG. 11F, expansion balloon 1004 is deflated and removed from within support casing 304.

Referring to FIG. 11G, inflatable cartridge 308 is inserted into support casing 304. Inflatable cartridge 308 includes inflatable structure 310, and inflatable structure 310 may be inflated using filling tool 526, as described above.

Referring to FIG. 11H, when expanded, inflatable structure 310 pushes out of apertures 602 and out of supporting casing 304, forming multiple protuberances 604 that may contact pedicle 1104. These protuberances 604 further anchor inflatable structure 310 to pedicle 1104, ensuring that neither inflatable structure 310 nor support casing 304 moves relative to vertebra 102.

In some embodiments, inflatable cartridge 308 may be adapted to accommodate various support casings 304. For instance, if support casing 304 includes several apertures 602, inflatable cartridge 308 may also include corresponding apertures (not shown), such that these apertures are radially aligned with apertures 602 when inflatable cartridge 308 is slideably inserted into support casing 304. As such, when inflatable cartridge 308 is inflated, 310 may pass through both sets of apertures to firmly anchor both inflatable cartridge 308 and support casing 304 into vertebra 102.

Generally, device 300 is depicted as being inserted laterally into vertebra 102. In some implementations, device 300 is inserted medially into vertebra 102. In some implementations, multiple devices 300 are each inserted laterally or medially into vertebra 102. Upon inflation of each device 300, devices 300 abut against each other, further stabilizing each device 300 within vertebra 102.

### Alternate Embodiment

Other example embodiments of a surgical implant device for strengthening and stabilizing a human vertebra, and a system for implanting the device in a human vertebra are contemplated. In some embodiments, the system includes a vertebral body access device, a stent, and an expandable liner.

The access device consists of a guide needle with a sharp pointed stylet. These may be made of stainless steel or any other suitable biocompatible metal.

The stent is generally tubular, having a central longitudinal channel for restraining the proximal segment of the inflatable liner. The stent has a proximal aperture with a coupling member mounted thereon for receiving a cement delivery nozzle, and a distal aperture that forms an outlet for the unrestricted segment of the inflatable liner. It also anchors the distal inflatable component to the rigid stent. The stent also has multiple side apertures allowing limited protrusion of the liner for more secure fixation of the stent to the surrounding bone. The stent may be made of stainless steel. In some embodiments, the stent may be made of other materials, such as a polymer, fabric, or composite material.

The liner is restrained within the stent and when inflated protrudes through multiple wall apertures to strengthen the interlock between the stent and the bone. In some embodiments, matching the liner protrusions to the shape and size of the side openings is incorporated during the manufacturing process of the liner using a correspondingly shaped mold. Using this mold, the balloon preform may be blown and stretched to form the protrusions. Through a mold heat set process, biaxial orientation of the noncompliant polymer can be achieved.

In some embodiments, limited compliance of the liner material is desirable, such that upon inflation of the liner inside the stent, the protrusions extend outward to the limited degree. Balloon features utilized in prior art medical balloons, such as those utilized in angioplasty balloons, may be incorporated in this invention. The liner may be compliant, semi-compliant or non-compliant. The wall thickness and compliance of some portions of the liner may differ from that of other portions. The liner may consist of a plurality of layers, and may be fiber reinforced in some areas and not in others to impart selective areas of noncompliance or forced bending or forced directional expansion of the liner.

The liner may be made of various materials, such as polyethylene terephthalate (PET), polyethylene, polyetheretherketone (PEEK), Pebax, Teflon, or other polyolefins.

In some embodiments, the liner that extends beyond the distal tip of the stent is unrestrained. In other embodiments, this segment is restrained by a scaffold that selectively restrains outward expansion of the liner and biases the expanding implant towards longitudinal directional expansion to achieve height and angle restoration of the fractured vertebra. When the liner lumen is cement impregnated and inflated, it becomes integral with the stent. In some embodiments, the scaffold is made of a fabric.

In an example implementation, an implantation procedure is carried out under local anesthesia and conscious sedation. A guide pin and/or drill is utilized to create a path in the trajectory of the guide needle to the anterior third of the vertebral body. The pin and/or drill is inserted in a postero-lateral approach under imaging observation, such as fluoroscopy. Alternatively, a unipedicular or bipedicular approach may be utilized to advance the needle with a sharp-tipped stylet into the posterior third of the fractured vertebral body. The stylet is then removed. A small amount of cement may be injected into the cavity to affect a degree of penetration of cement into interstices of the cancellous bone structure.

The inflation cannula is primed with cement and its delivery nozzle is connected to the proximal filling port of the stent. The cement delivery nozzle has male threads that couple with female threads of the connector member. The inflation cannula, with the stent coupled to its tip, is then inserted through the access needle and advanced to its tip. The access needles, with the stent therein, is then advanced to the anterior third of the vertebral body. While holding the inflation cannula fixed in position, the user then withdraws the guide needle, deploying the stent in a position which extends from the posterior margin of the pedicle to the anterior third of the vertebral body. The implant is then inflated. An obturator is then introduced to the tip of the delivery nozzle to expel any remaining cement and to insure proper disengagement after the cement hardens. After the cement hardens, the cement delivery nozzle is disconnected and removed, together with the guide needle.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described components and systems can generally be integrated together in a single product or packaged into multiple products.

Thus, particular embodiments of the subject matter have been described. Other embodiments are within the scope of the following claims.

What is claimed is:

1. An apparatus comprising:
an axially extending cannula, comprising:
a first end, forming a first access hole along the axial extension of the cannula;
a second end having an internally threaded portion, forming a second access hole along the axial extension of the cannula; and
a tubular channel extending from the first access hole to the second access hole;
an axially extending support device comprising:
a first end having an externally threaded portion, forming a first access hole along the axial extension of the support device; and
a second end, forming a second access hole along the axial extension of the support device;
a tubular channel extending from the first access hole to the second access hole;
wherein the threaded portion of first end of the support device is adapted to cooperatively fit into the threaded portion of the second end of the cannula; and
wherein an internal diameter of the support device is smaller than an internal diameter of the cannula; and
an implant cartridge comprising:
a tubular liner element having a first portion, forming a first access hole, and a second portion, forming a second access hole, where an external diameter of the first portion corresponds to the internal diameter of the cannula, and an external diameter of the second portion corresponds to the internal diameter of the support device; and
an inflatable structure within the liner element, having a filling aperture;
wherein the implant cartridge is adapted to fit within the cannula and the support device when the cannula and the support device are assembled;
wherein when the cannula, support device, and implant cartridge are assembled, filling the inflatable structure with a material expands the balloon out of the second access hole of the support device.

2. An apparatus comprising:
an axially extending tubular cannula having an internally threaded end portion;
an axially extending tubular support device having an externally threaded end portion;
an inflatable structure adapted to fit within the cannula and the support device when the apparatus is assembled;
wherein when the apparatus is assembled, the threaded end portion of the support device couples into the threaded end portion of the cannula; and
wherein filling the inflatable structure with a material expands the inflatable structure out of the support device.

3. The apparatus of claim 2, wherein an internal diameter of the support element is smaller than an internal diameter of the cannula.

4. The apparatus of claim 2, further comprising a delivery device adapted to transfer a material from the delivery device to the inflatable structure.

5. The apparatus of claim 2, further comprising a guide device having a pointed end, adapted to fit through the cannula and through the support device when cannula and support device are assembled.

6. The apparatus of claim 2, wherein the apparatus is of a surgically compatible material.

7. The apparatus of claim 2, wherein portions of the apparatus are of a biocompatible material.

8. The apparatus of claim 2, wherein portions of the apparatus are of a radiopaque material.

9. The apparatus of claim 2, wherein the apparatus is adapted to insert into a vertebra.

10. The apparatus of claim 9, wherein the apparatus adapted to implant the inflatable structure within a vertebra.

11. The apparatus of claim 10, wherein the inflatable structure strengthens the vertebra when inflated.

12. The apparatus of claim 10, wherein the inflatable structure strengthens the vertebra when inflated.

13. The apparatus of claim 9, wherein the cannula may be released from the access device after the apparatus is inserted into a vertebra.

14. The device of claim 4, wherein the material may be poly methyl mecratylate, silicone, bone cement, epoxy, acrylic, or a combination thereof.

15. The device of claim 4, wherein the material cures or reacts within the inflatable structure such that the substance changes phase, tensile strength, density, size or other physical properties upon curing or reacting.

16. A method comprising:
attaching a cannula to a support device by screwing a threaded portion of the cannula to a threaded portion of the support device;
inserting the support device into the body
inserting an implant cartridge into the cannula and support device, the implant cartridge comprising an inflatable structure;
inflating the inflatable structure such that a portion of the inflatable structure exits the support device and into the body;
removing the cannula from the support device by unscrewing the threaded portion of the cannula from the threaded portion of the support device;
wherein after removing the cannula, the support device and the inflatable structure remain in the body.

17. The method of claim 4, further comprising inserting a guide device into the cannula and support device prior to inserting the support device into the body, and removing the guide device after inserting the support device into the body.
